(19) United States
(12) Patent Application Publication
(10) Pub. No.: US 2025/0057517 A1
(43) Pub. Date: Feb. 20, 2025

(54) CALIBRATION BODY, MEDICAL ASSISTANCE SYSTEM, METHOD FOR EVALUATING A CALIBRATION AND COMPUTER-READABLE STORAGE MEDIUM

(71) Applicant: B. Braun New Ventures GmbH, Freiburg (DE)

(72) Inventor: Jean Stawiaski, Freiburg (DE)

(21) Appl. No.: 18/799,054

(22) Filed: Aug. 9, 2024

(30) Foreign Application Priority Data
Aug. 18, 2023 (DE) 10 2023 122 203.1

(51) Int. Cl. A61B 17/00; A61B 34/20; A61B 34/30; A61B 90/00; A61B 90/96

(52) U.S. Cl. CPC A61B 17/00; A61B 34/30; A61B 90/96; A61B 2017/00725; A61B 2034/2055; A61B 2034/2065; A61B 2034/2072; A61B 2090/3937; A61B 2090/3979; A61B 2090/3995

(57) ABSTRACT

A calibration body for a medical assistance system includes an optical marker as a first marker. The optical marker is detectable by a movably arranged optical recording unit of the assistance system with a first recording, on the basis of which a first layer of the calibration body is determinable relative to the first optical recording system. The calibration body also includes an IR marker arrangement as a second marker. The IR marker arrangement is detectable by an IR tracking unit of the assistance system with a second recording, on the basis of which a second layer of the calibration body is determinable relative to the IR tracking unit, to evaluate at least one calibration of the assistance system via the optical marker and the IR marker arrangement. A medical assistance system, computer-implemented method for evaluation, and computer-readable storage medium can include or be used with the calibration body.

### CROSS-REFERENCE TO RELATED APPLICATION

This application claims priority under 35 U.S.C. § 119 to German Application No. 10 2023 122 203.1, filed on Aug. 18, 2023, the content of which is incorporated by reference herein in its entirety.

### FIELD

The present disclosure relates to a calibration body by means of the detection (recording) of which at least one calibration of a surgical assistance system can be evaluated. In addition, the present disclosure relates to a surgical assistance system, a method, in particular a computer-implemented method, for the evaluating at least one calibration of the assistance system, in particular a hand-eye calibration, as well as a computer-readable storage medium and computer program.

### BACKGROUND

In a number of robot-assisted surgical interventions, such as neurosurgery or spinal surgery, microscopic and endoscopic visualization or imaging and navigation of an instrument in the surgical intervention area are essential, operation-critical technologies. Both technological modalities, namely visualization on the one hand and navigation on the other, enable the surgeon to target, safely and precisely identify and to manipulate a tissue, as well as to precisely reference the tissue on preoperative recordings of the tissue. Even a millimeter of deviation can lead to a success or failure of the intervention.

A surgical assistance system typically has an optical recording unit, for example a surgical microscope or endoscope, which is connected to a movable robot arm and takes images of the surgical area and provides them to the surgeon on a display unit, for example a monitor. An infrared (IR) tracking camera of the assistance system continuously traces/tracks an IR rigid body rigidly coupled to the recording unit, so that a data processing unit can determine the position of the recording unit relative to the local coordinate system of the IR camera based on a recording of the IR camera.

To determine the correct position within the assistance system, the rigid body requires calibration; the internal optical calibration of the recording system with regard to its distortion coefficient, its internal projection matrix and possibly its external transformation matrix in the case of a stereo camera; the so-called hand-eye calibration of the recording system with reference to the IR tracking camera and the hand-eye calibration of the system with reference to the IR tracking camera. Since these calibrations and the resulting global precision of the navigation can be disrupted by external influences, such as shocks, mechanical deformation, etc., it is necessary to be able to check or evaluate the assistance system before or during an intervention with regard to its calibration.

According to the state of the art, individual evaluation devices are used to evaluate the large number of calibrations mentioned above, so that the evaluation of the calibrations involves considerable effort for the production, maintenance and use of the evaluation devices.

### SUMMARY

Therefore, the tasks and objectives of the present disclosure are to avoid or at least mitigate the disadvantages of the prior art and to provide a calibration body, a surgical assistance system, a method for evaluation and a storage medium, which in each case allows a reliable and secure evaluation of a calibration.

A subtask with regard to the surgical assistance system can be seen in ensuring the evaluation of the calibration or calibrations with the least possible device and process engineering effort.

The tasks are solved with respect to a calibration body according to the present disclosure, with respect to a surgical assistance system according to the present disclosure, with respect to a method according to the present disclosure, and with respect to a computer-readable storage medium according to the present disclosure.

In principle, a calibration body with different markers (one could also say different marker detection modalities) is provided, each of which is intended to be detected by one of a several recording units of the assistance system to be calibrated. The following are particularly suitable recording units: an optical recording unit for displaying the intervention area and an infrared tracking camera for tracking IR markers or IR rigid bodies rigidly coupled to the optical recording unit. In this way, a common calibration body is provided for evaluating different calibrations, by means of which the quality or quality of at least one calibration of the assistance system, in particular the hand-eye calibration of the navigated, robotically actuated optical recording system, can be evaluated. The calibration body can be used to evaluate or verify the accuracy of an internal calibration of the optical recording system (surgical microscope, surgical endoscope) and its hand-eye calibration. The calibration body can be used to evaluate the calibration of surgical assistance systems that have one or more (optical) recording units or (IR) tracking units with fixed or with variable focal length and magnification.

A preferably rigid, calibration body according to the disclosure is intended for a navigated medical, in particular surgical, assistance system and has an optical marker as the first marker. The optical marker is adapted to be detected by a preferably movable arrangeable optical recording unit of the assistance system with a first recording, so that a first layer, in particular a first transformation, of the calibration body relative to the first optical recording system, in particular to its local, first coordinate system, can be determined on the basis of the first image, in particular by means of a machine vision. Furthermore, the calibration body has an infrared (IR) marker arrangement as a second marker. The IR marker arrangement has at least three, preferably four, IR markers and is adapted to be captured by an IR tracking unit of the assistance system, in particular by an IR tracking camera, with a second recording, so that on the basis of the second recording, in particular by means of machine vision, a second position, in particular a second transformation, of the calibration body relative to the IR tracking unit, in particular to its local, second coordinate system, can be determined. By the first and second markers, i.e. the optical marker and the IR marker arrangement, prepare the calibration body for detection by different recording units of the assistance system and thus enable the evaluation of several of the above-mentioned calibrations, with less equipment-related and process-related engineering effort compared to conventional solutions.

Advantageous embodiments are explained in particular below.

In one variant, the rigid calibration body is formed of one part or one piece. In this way, the geometric precision over time is particularly high. Alternatively, in another variant, it is joined or connected from a plurality of parts in order to form one part, in order to make the manufacturing process efficient and also to be designed to be modular and interchangeable.

The number of markers of the calibration body to be captured is of course not limited to two (first markers, second markers). Depending on the need, a third marker can, for example, be additionally provided.

According to a further advancement, the first or optical marker is formed as an optical pattern. This can be formed at least in sections by a flat bar code or barcode, grid code or matrix code, QR code, laser print or imprint. The optical pattern can be formed by a planar anodizing or by an etching or engraving. Additional data can be read out by, for example, a QR code.

According to a preferred further advancement, the optical marker has, preferably four, optical patterns that are preferably arranged in one plane and/or are respectively arranged symmetrically to one another for two orthogonal axes, in particular are arranged as corners of a rectangle. This means that there are enough markers spaced apart from each other to record the position, which can be captured with a single recording.

According to a preferred further advancement, the IR marker arrangement, preferably four, has infrared reflector bodies and/or IR LEDs, which are preferably arranged in one plane and/or are respectively arranged symmetrically to one another for two orthogonal axes, in particular arranged as corners of a rectangle.

A further advancement in which the optical patterns of the first marker are arranged in a first plane and the infrared reflector bodies and/or IR LEDs are arranged in a second plane is advantageous for the detectability of the first and the second marker. The first plane is preferably parallel to the second plane.

Preferably, in a plan view of the calibration body, in particular in a plan view perpendicular to the first or second plane, the optical patterns are arranged within the infrared reflector bodies and/or IR LEDs. In other words, the second marker with its infrared reflector bodies and/or IR LEDs covers a larger area than the first marker with its optical patterns. As a result, the first and second markers are in particular adapted to be optimally captured from different distances and/or with different recording modalities. In other words, according to this embodiment, a characteristic length of the second marker, in particular a diagonal or a distance between the infrared reflector bodies and/or IR LEDs, is greater than a characteristic length of the first marker, in particular a diagonal or a distance of the optical patterns, wherein the first and second markers are particularly adapted for this purpose to be optimally detected from different distances and/or with different recording modalities.

According to a preferred embodiment, the calibration body has a base body on the surface of which—preferably formed by the first plane—the optical or first marker extends. The infrared reflector bodies and/or IR LEDs of the second marker are preferably each arranged on an extension, boom, or arm projecting from the base body.

According to a particularly preferred further advancement, which allows an evaluation on the basis of a human, visual inspection, the first or optical marker is supplemented by at least one optically detectable geometric feature of the calibration body itself, in particular the base body. The optically detectable geometric feature is preferably a body edge and/or a depression or groove and/or an elevation or rib, which in particular lies on an axis of symmetry of the base body.

In particular, in order to enable and simplify the manual and/or mechanical holding and/or positioning of the calibration body in the respective field of view of the optical recording system and the IR tracking unit, the base body comprises according to a further advancement an undercut, in particular an approximately anvil-shaped thickening, on its posterior side with respect to the markers. One can also say that in particular a cross-section of the base body is designed in the form of a cross-section of an anvil (in the manner of a contour of a cantilevered chef's hat).

According to a preferred further advancement, the IR reflector bodies or IR LEDs are arranged such that they are protruding from a surface of the base body comprising the optical markers or the optical patterns, in particular vertically protruding, for example by being arranged on bolts or ribs that protrude above the surface. In this way, the aforementioned arrangement of the markers on planes spaced apart from one another, realizes in particular the first plane and the second plane.

The surface of the base body comprising the optical marker or the optical pattern preferably comprises crossed grooves or grooves, in particular grooves or channels extending orthogonally to one another, as geometric features.

In particular for the secure and variable fastening of the calibration body to a holding device, the calibration body comprises, preferably its base body, recesses or through-recesses. These extend in particular transversely to a longitudinal direction of the base body.

For the same reason, the calibration body, preferably its base body, comprises a bore hole in the longitudinal direction, in particular a through-bore, particularly preferably a plurality of through-bores.

According to a further advancement, the optical marker extends over more than only one side of the calibration body, in particular the base body, and/or is supplemented by geometric features on more than one side of the calibration body, in particular the base body. Preferably, as such an additional geometric feature, a series of bores with a diameter decreasing or increasing in the direction of the row is formed on one end face of the base body, which enable an evaluation of the calibration body from a different viewing direction.

In terms of detectability, it is fundamentally advantageous if the optical patterns are designed with the greatest possible contrast. A two-tone pattern proves to be simple and advantageous. The optical pattern formed in particular from complementary colors is preferred due to the resulting strongest contrast. In particular, white and black or a light color or color perception and a dark color or color perception should be mentioned here. For example, a light color can be created by keeping the surface structure “smooth”, while an engraving or corrugation or roughening of a surface, on the other hand, causes partial absorption of light and is therefore perceived as dark. This allows an optical pattern to be created sustainably by processing a surface without applying a color. One of the colors, and thus a region of the optical pattern, can be formed, for example, by the surface of the calibration body that is not treated separately in terms of color. The other color, and thus the other region of the optical pattern, can be designed in complementary colors, for example by means of printing, laser printing, etching or another surface treatment.

The term “position” refers to a geometric position in three-dimensional space, which is specified in particular by means of coordinates of a Cartesian coordinate system. In particular, the position can be specified by the three coordinates X, Y and Z.

The term “orientation” in turn indicates an orientation (such as position) in space. It can also be said that the orientation indicates a direction or rotation in three-dimensional space. In particular, the orientation can be specified using three angles.

The term “condition” comprises both a position and an orientation. In particular, the condition can be specified using six coordinates, three position coordinates X, Y and Z and three angular coordinates for the orientation.

The term “transformation” encompasses a relative position and a relative orientation with reference to a coordinate system and, in the context of this disclosure, preferably means a “6D transformation” which encompasses the three coordinates X, Y, Z for the relative position, as well as the three angular coordinates for the relative orientation.

The tasks of the present disclosure are solved with respect to a surgical assistance system for use in a surgical intervention on a patient in that the surgical assistance system comprises:
an optical recording unit, in particular with a surgical microscope and/or a surgical endoscope, which is connected to a movable end segment of a robot arm of a robot and which is adapted to create and provide a first recording, preferably of a surgical area;
an IR tracking unit, in particular an IR tracking camera, which is adapted to create and provide a second recording that contains an IR rigid body fixed rigidly to the end segment and preferably contains the surgical area. According to the present disclosure, the assistance system also has:
a calibration body according to at least one aspect of the preceding disclosure and a data processing unit, in which preferably an algorithm of machine vision is stored for execution, and which is adapted to:
determine on the basis of the first image of the optical marker a first position of the calibration body relative to the first optical recording unit, in particular a first transformation of the calibration body relative to a first local coordinate system of the first optical recording unit;
determine a second position of the calibration body relative to the IR tracking unit, in particular a second transformation of the calibration body relative to a second local coordinate system of the IR tracking unit, based on the second recording of the IR marker arrangement;
determine a rigid body transformation of the IR rigid body relative to the second local coordinate system of the IR tracking unit based on the second recording of the IR rigid body; and
determine a connection between the rigid body transformation, the hand-eye transformation of the first optical recording unit and the first transformation, in particular by means of a transformation matrix multiplication in a defined order, by increasing an already calibrated hand-eye transformation of the first optical recording unit relative to the rigid body, and to compare this connection with the second transformation, so that an evaluation of a calibration of the assistance system is carried out on the basis of the comparison.

According to a further advancement, the data processing unit is adapted to determine the link and to determine the second transformation for a spatial point shown in the first recording and in the second two recordings, which is preferably a point of the calibration body. In addition, according to the further advancement, the data processing unit is adapted to determine an Euclidean distance based on a difference between the determined link and the determined second transformation as a measure of a quality or goodness of the hand-eye transformation of the first optical recording unit relative to the rigid body, so that a hand-eye calibration of the first optical recording unit can be quantitatively evaluated by indicating this dimension.

According to a further advancement, the data processing unit is adapted to determine the Euclidean distance for at least two different poses or layers of the robot, in particular the end segment. In this case, the data processing unit according to the further advancement is further adapted to determine a difference in the Euclidean distances of the poses or positions as a measure of the rigid body transformation or the hand-eye transformation of the rigid body relative to the IR tracking unit, so that by specifying this measure, a hand-eye calibration of the robot can be quantitatively evaluated.

According to a further advancement, the assistance system also has a display unit that can be controlled by the data processing unit, in particular a screen or VR glasses, and the data processing unit is adapted to: detect or ascertain a presence of the calibration body in a respective field of view of the first optical recording unit and the IR tracking unit, in particular in the first recording and in the second recording. The presence is given in particular if the marker assigned to it is ascertained to be present in the respective recordings by means of the machine vision of the data processing unit. In the first recording, it is the presence of the first marker, in the second recording, it is the presence of the second marker. Furthermore, it is adapted, in the case of the ascertained presence, to determine the first transformation of the calibration body relative to the first coordinate system of the optical recording unit, in addition to back-project geometric features of the calibration body into a plane of the first recording on the basis of this first transformation and furthermore to control the display unit to display the first recording together with the back-projected geometric features.

This enables the user to detect a deviation of the back-projected geometric features from the underlying, real geometric features when looking at the first image and to qualitatively assess/evaluate the hand-eye calibration of the optical recording unit.

Preferably, the assistance system has a robot base as a local connection point of a robot comprising the above-mentioned robot arm. The robot arm is preferably movable and preferably has at least one articulated robot arm segment, in particular the said end segment.

With regard to a method for evaluating at least one calibration of a medical, in particular surgical, assistance system, preferably an assistance system according to the present disclosure, the objects and objectives of the present disclosure are achieved in that the method comprises steps: placing a calibration body, in particular according to at least one aspect of the preceding description, in a first field of view of an optical recording unit, in particular a surgical microscope and/or a surgical endoscope, which is connected to a movable end segment of a robot arm, and in a second field of view of an IR tracking unit, in particular an IR tracking camera;
producing and providing a first image of an optical marker of the calibration body by the optical recording unit; and
creating and providing a second image of an IR marker arrangement of the calibration body and an IR rigidly fastened to the end segment by the IR tracking unit; and
according to the present disclosure at least determining a first position of the calibration body relative to the optical recording unit, in particular determining a first transformation of the calibration body relative to a first local coordinate system of the optical recording unit, on the basis of the first recording, by a data processing unit of the assistance system in which a machine vision algorithm is stored for execution for the purpose of determining positions and/or transformations; and
determining a second position of the calibration body relative to the IR tracking unit, in particular determining a second transformation of the calibration body relative to a second, local coordinate system of the IR tracking unit, on the basis of the second recording, by the data processing unit; and
evaluating at least one calibration of the assistance system based on at least the first transformation and the second transformation.

Preferably, the method has a step of detecting a presence of the calibration body in a respective field of view of the first optical recording unit and the second IR tracking unit, in particular in the first recording and in the second recording, by the data processing unit on the basis of the first and the second recording.

Depending on the detected presence, according to a further advancement of the method, at least one evaluation can be started or carried out automatically and/or according to a further development of the method, the user or operator can be asked whether an evaluation should be carried out.

According to a further advancement of the method, which allows a quantitative evaluation of a hand-eye calibration of the first optical recording unit, the method has the following steps:
determining a link of transformations, starting from the local, second coordinate system of the IR tracking unit, via the rigid body, the optical recording unit, to the calibration body. In other words: determining a link from a rigid body transformation of the rigid body relative to the second coordinate system, from a transformation of the optical recording unit relative to the rigid body and from the first transformation of the calibration body relative to the first coordinate system of the optical recording unit, by the data processing unit, in each case for at least one spatial point which is imaged both in the first recording and in the second recording;
determining the second transformation for the at least one space point, by the data processing unit;
determining an Euclidean distance based on a difference between the determined link and the determined second transformation as a measure for a hand-eye calibration of the optical recording unit; and
evaluating quantitatively the hand-eye calibration of the optical recording unit based on the Euclidean distance, by the data processing unit.

According to a further advancement of the method, which allows a quantitative evaluation of a hand-eye calibration of the robot, the method has further steps:
determining the Euclidean distance for different poses or layers of the robot, in particular the end segment, by the data processing unit;
determining a difference of the Euclidean distances of the poses or layers as a measure of the hand-eye calibration of the robot, by the data processing unit; and
evaluating quantitatively the hand-eye calibration of the robot based on the difference of the Euclidean distances of the poses, by the data processing unit.

In a further development, the method has a step of performing a new calibration and/or issuing an error message, if the quantitative evaluation of at least one of the hand-eye calibrations is negative, in particular if the specific Euclidean distance or the specific difference of the Euclidean distances of the layers or poses lies above a respective predetermined threshold stored in the data processing unit.

In order to enable an intuitive, visual assessment of the quality or goodness or accuracy of a calibration of the assistance system, for example by the user or surgeon, the method comprises the following steps according to a further advancement:
detecting the presence of the calibration body in the respective field of view of the optical recording unit and the IR tracking unit, in particular in the first recording and in the second recording, by the data processing unit on the basis of the first and the second recording; and in the case of presence
determining the first transformation of the calibration body relative to the first coordinate system of the optical recording unit, by the data processing unit;
determining a back projection of at least one geometric feature of the calibration body into a plane of the first recording, at least on the basis of the determined first transformation, by the data processing unit;
controlling the display unit to display the first recording together with the at least one back-projected geometric feature, by the data processing unit; and
evaluating qualitatively or assessing qualitatively the calibration by a surgeon by visually comparing and evaluating the at least one geometric feature that is back-projected into the first image with the geometric feature visible in the first image with regard to a shape and/or positional deviation.

If the surgeon comes to a negative result, for example if the shape deviation and/or the position deviation exceeds his personal limit, he can initiate the calibration in a subsequent step of the method.

With respect to a computer-readable storage medium and with respect of a computer program, the tasks and objectives of the present disclosure are solved in that the computer-readable storage medium or rather the computer program comprises instructions which, when executed by a computer, cause the computer to perform the method steps of the method for evaluation according to the present disclosure.

The features of the calibration body of the present disclosure and the surgical assistance system of the present disclosure may be interchanged.

### BRIEF DESCRIPTION OF THE DRAWINGS

The present disclosure is explained below with reference to preferred embodiments with the aid of accompanying figures.

FIG. 1 shows a perspective view of a calibration body, according to an exemplary embodiment;

FIG. 2 shows a surgical assistance system with a robot arm of a robot, an optical recording unit and an IR tracking unit, each with a view of the calibration body according to FIG. 1, and with transformation paths used for the quantitative evaluation of a hand-eye calibration of the recording unit, according to one exemplary embodiment;

FIG. 3 shows the surgical assistance system according to FIG. 2 with transformation paths used for the quantitative evaluation of a hand-eye calibration of the robot;

FIG. 4 shows the surgical assistance system according to FIGS. 2 and 3 for the transformation paths used for the qualitative evaluation of a calibration of the assistance system;

FIG. 5 shows a recording provided by the optical recording system, in which the recorded calibration bodies and mathematically determined geometric features of the calibration body are superimposed for the qualitative evaluation of the calibration, in the event, the calibration is of poor quality;

FIG. 6 shows a recording provided by the optical recording system, in which the recorded calibration bodies and mathematically determined geometric features of the calibration body are superimposed for the qualitative evaluation of the calibration, in the event, the calibration is of high quality; and

FIG. 7 shows a method for evaluating a calibration of the assistance system according to FIGS. 2 through 4, according to an exemplary embodiment.

The figures are merely schematic in nature and are only intended to aid understanding of the publication. The features of the various embodiments can be interchanged.

### DETAILED DESCRIPTION

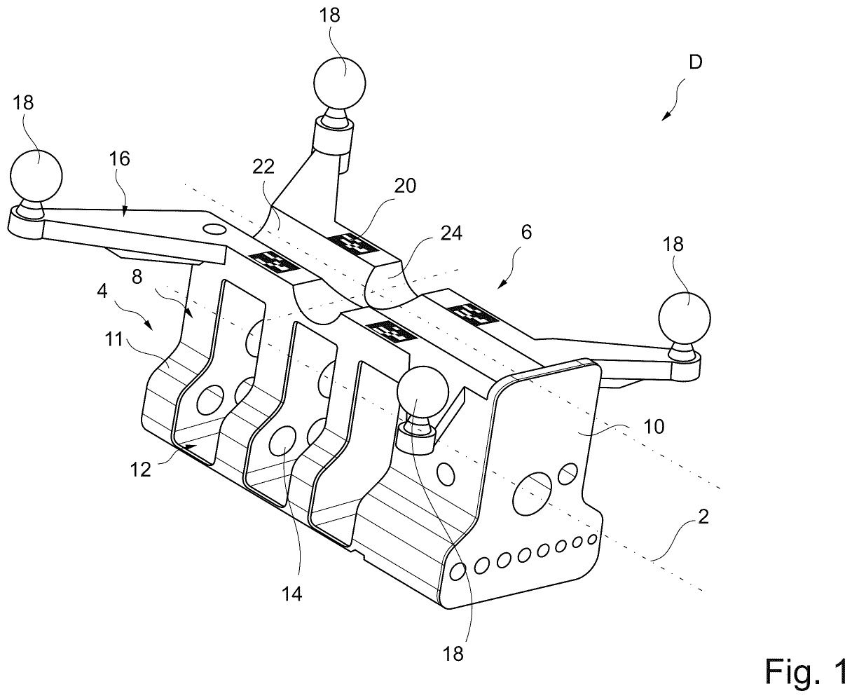

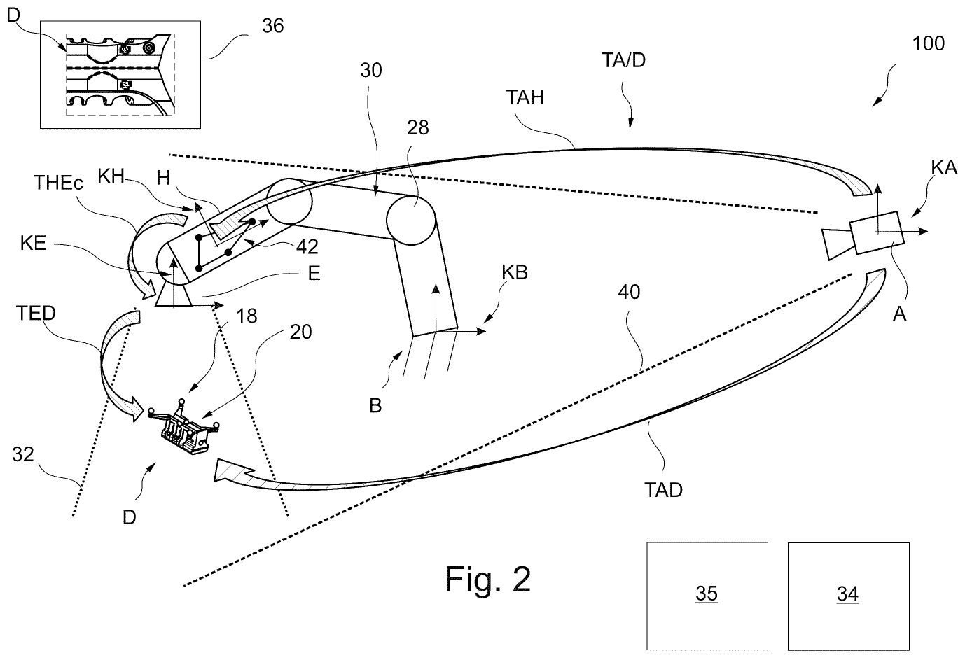

FIG. 1 shows a perspective view of a rigid calibration body D, which is intended to be used for the evaluation of a calibration of a surgical assistance system (cf. FIG. 2 et seq.) or one of its subcomponents. The calibration body D has a base body 4, which extends substantially cylindrically along a longitudinal axis 2, with a top side 6, edge side 8 and face side 10 facing the observer in FIG. 1. For better handling, for example for gripping by a surgeon or a suitably adapted robot hand, the base body 4 is designed with its lower section thickened, similar to an anvil. In this way, an undercut 11 is formed, which makes the calibration body D easier to grip.

For variable fastening, for example on a holding device, the base body 4 is also perforated by three through-recesses 12 with a substantially rectangular cross-section extending from the edge side 8 to the opposite edge side. The three through-recesses 12 are arranged unevenly distributed in the direction of the longitudinal axis 2, in that they have a variation with respect to their distance from one another, and in that their distribution along the longitudinal axis 2 is irregular, so that the group of through-recesses 12 is arranged closer to the rear face than the face 10.

In the direction of the longitudinal axis 2 and concentric to it, the base body 4 is penetrated by a through-bore 14, which offers a first possibility for pivotal mounting of the calibration body D. Furthermore, the base body 4 is penetrated in the region of the lower-sided, anvil-shaped section, starting from the rear face side into the second of the through-recesses 12, from the through-bores 14 running side by side, which for example represent an alternative possibility of a pivot axis of the calibration body D or via which the calibration body D can be locked, for example at a defined pivotal angle.

According to FIG. 1, a first optical marker is arranged on a level surface of the top side 6 in the configuration of four flat optical patterns 20. The four optical patterns 20 are designed as an individual matrix code and extend in an axially symmetrical arrangement on both sides of a longitudinal groove 22 with a V-shaped cross-section and a transverse groove 24 with a U-shaped cross-section, which are formed in the surface of the top side 6.

The first marker 20 thus formed is provided to be detected by an optical recording unit of a surgical assistance system by means of its first recording, and that on the basis of the first recording by a data processing unit of the assistance system a first position, or a first transformation, of the calibration body D relative to the optical recording unit and thus to its local, first coordinate system can be determined (cf. FIG. 2 et seq.).

According to FIG. 1, at the upper corners of the base body 4, boom 16 protrudes, approximately in an x-shaped arrangement. A spherical infrared reflector body 18 projects perpendicularly from the outside laying end sections of the booms 16 in the direction away from the top side 6. The four IR reflector bodies 18 form an IR marker arrangement as second marker of the calibration body D.

The second marker 18 thus formed is provided for it to be detected by an IR tracking unit of the surgical assistance system by means of its second recording, and that on the basis of the second recording by the data processing unit of the assistance system a second position, or a second transformation, of the calibration body D relative to the IR tracking unit and thus to its local, second coordinate system can be determined (cf. FIG. 2 et seq.).

With the first marker 18 and second marker 20 arranged on the (only) calibration body D, the calibration body D can be detected by different recording units of the assistance system. The optical patterns 20 of the first marker can be captured by the optical recording unit of the assistance system, such as an operating microscope or endoscope, and the IR reflector bodies 18 of the second marker can be detected by the IR tracking unit of the assistance system, such as an IR tracking camera. In this way, the calibration body D according to the present disclosure offers different paths for its detection, so that results of these different detections can be compared and, based on this comparison, at least one calibration of the assistance system can be evaluated without having to resort to several different calibration bodies (see FIG. 2 et seq.).

FIG. 2 shows the already mentioned medical, in particular surgical, assistance system 100 for a surgical intervention, according to a preferred exemplary embodiment. The assistance system 100 has an optical recording unit designed as an operations microscope E. Alternatively or additionally, the optical recording unit can be or comprises, for example, an operation endoscope. The surgical microscope E is connected to a movable end segment H of a robot arm 30 of a robot, the base of which is arranged in a fixed position at least for a limited time, in particular for evaluation.

The surgical microscope E creates a first image within its first field of view 32 and provides it to a data processing unit 34 of the assistance system 100, which controls a display unit of the assistance system 100 designed as a monitor 36 to display the first image. The data processing unit 34 has an algorithm for machine vision stored for execution, by which it can determine a first layer or first transformation TED of the calibration body D with reference to a local coordinate system KE of the surgical microscope E based on the first image and the optical pattern 20 shown therein.

For navigation purposes, the assistance system 100 has an IR tracking unit designed as an IR tracking camera A and an IR rigid body 42 is rigidly fastened to the end segment H, to which the optical recording unit E is rigidly connected.

The IR tracking camera A creates a second image of the entire scene with the robot arm 30, the end segment H, the rigid body 42, the optical recording unit E and the calibration body D within its second field of view 40 and provides this second image to the data processing unit 34 for analysis.

The data processing unit 34 analyzes the second recording with the rigid body 42 shown therein and the IR marker arrangement 18 shown therein by means of the machine vision algorithm and thereby determines—in each case with reference to the local coordinate system KA of the IR tracking camera A—a position or transformation TAH of the end segment H, as well as a second position or second transformation TAD of the calibration body D.

In particular rigid, transformation, a THEc of the optical recording unit E with reference to the local coordinate system KH of the end segment H of the robot arm 30 is stored in the data processing unit 34.

This transformation THEc is preferably a 6D-transformation and can in particular be referred to as a hand-eye calibration of the optical recording unit E. It depends in particular on inner optical calibration parameters of the optical recording unit E, such as a distortion coefficient, an inner projection matrix and an outer transformation matrix. It is preferably estimated or stored in a predetermined manner.

In the event that the optical recording unit E is equipped with a variable focus and zoom, a respective transformation THEc is estimated or predetermined for each focus and zoom. The quantity of these estimated or predetermined transformations THEc represent the hand-eye calibration parameters of the optical recording unit E.

From the local coordinate system KA of the IR tracking camera A to the calibration body D, two independent transformation or calibration paths are thus determined in the data processing unit 34: a direct path TAD, directly represented by the second position or second transformation TAD of the calibration body D relative to the coordinate system KA and an indirect path TA/D via the robot arm 30 and the optical recording unit E. The indirect path TA/D is represented by a link between the transformation TAH of the end segment H to the local coordinate system KA and the transformation THEc of the optical recording unit E to the local coordinate system KH and the transformation TED of the calibration body D to the local coordinate system KE.

In the following, the two paths TAD and TA/D can be used for quantitative and qualitative evaluation of at least one calibration of the assistance system 100. The preferred evaluated types of calibration are the hand-eye calibration of the optical recording unit E and the hand-eye calibration of the robot.

The quantitative evaluation of the hand-eye calibration or calibration parameters of the optical recording unit E is carried out according to FIG. 2 in such a way that the calibration body D is first placed in the field of view 32 of the optical recording unit E.

The data processing unit 34 determines the presence of the calibration body D in the first image and in the second image and starts the evaluation of the hand-eye calibration of the optical recording unit E.

For this purpose, the combination of the transformations TAH, THEc and TED is first equated with the second transformation TAD by the data processing unit 34.

$$TA / D = TAD \qquad ( 1 )$$

$$TAH * THEc * TED = TAD \qquad ( 2 )$$

with TAH as a transformation of the end segment H to the local coordinate system KA of the IR tracking camera A, THEc as a transformation of the optical recording unit E to the local coordinate system KH of the end segment H, TED as the first transformation of the calibration body D to the local coordinate system KE of the optical recording unit E, and TAD as a second transformation of the calibration body D to the local coordinate system KA of the IR tracking camera A.

Using equation (2), the data processing unit 34 determines a Euclidean error and, as disclosed, assigns this error to the transformation THEc of the optical recording unit E relative to the local coordinate system KH of the end segment H—and thus to the hand-eye calibration of the optical recording unit E.

The Euclidean error is obviously defined as Euclidean distance E. To determine the Euclidean distance E, the data processing unit 34 uses at least one spatial point x, preferably a body point x of the calibration body D, and determines for this spatial point x both the connection TA/D of the above-mentioned transformations and the transformation TAD.

The Euclidean distance is then ascertained with equation (3) for:

$$E ( x ) = IITA / D ( x ) - TAD ( x ) II \hat{} 2 \qquad ( 3 )$$

According to the disclosure, the Euclidean error/Euclidean distance E(x) is defined as a measure of the quality or goodness of the calibration and/or the calibration parameters of the hand-eye calibration of the optical recording unit E.

If the determined Euclidean error/the determined Euclidean distance E(x) exceeds a predetermined limit stored in the data processing unit 34, at least an error message or warning is issued that a recalibration is necessary, so that a user or operator can preferably initiate and/or terminate the recalibration as required.

In addition, the Euclidean error/Euclidean distance E can be determined for several spatial points x.

In addition, a statistics of the Euclidean error/Euclidean distance can be evaluated to obtain a more detailed report of accuracy, such as a mean error, a maximum error, a median, and the like.

Alternatively, the Euclidean error/the Euclidean distance E is used as a measure for the quality or goodness of a global calibration of the assistance system 100, instead of—as described above—solely as a measure for the quality or goodness of the hand-eye calibration of the optical recording unit E.

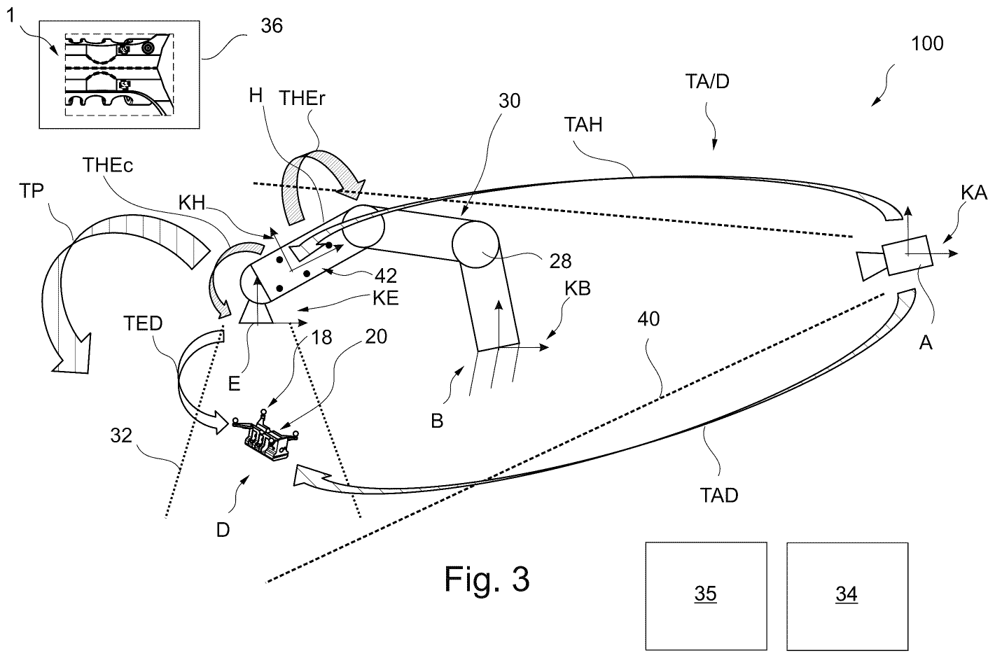

Following the above-described hand-eye calibration of the optical recording unit E, the quantitative evaluation of the hand-eye calibration of the robot H, 30 can be carried out, which is described in more detail with reference to FIG. 3.

According to FIG. 3, a, in particular rigid, transformation THEr of the end segment H with reference to a maximum proximal joint 28 of the robot arm 30, and thus to the base of the robot, is stored in the data processing unit 34. Preferably, this transformation THEr is estimated or predetermined. The transformation THEr is also referred to as a hand-eye calibration of the robot H, 30.

According to FIG. 3, a control unit 35 of the assistance system 100 controls the robot arm 30 in such a way that it assumes different, consecutive positions or poses, which is represented in FIG. 3 by the arrow TP. The control is carried out in such a way that it is ensured that the calibration body D is in the assumed position or pose and, when the first and second images are taken, is within the first field of view 32 and the second field of view 40. The data processing unit 34 determines the presence of the calibration body D in the first and second images and starts the evaluation of the hand-eye calibration of the robot H, 30.

The above equation (3) is also used for the evaluation of the hand-eye calibration THEr of the robot H, 30 by determining the Euclidean error/the Euclidean distance E(x) for each position or pose of the robot for the respective spatial point x and storing it in the data processing unit 34.

A difference between the Euclidean errors/Euclidean distances E(x) of two different positions or poses then represents, in a variant of the evaluation according to the disclosure, a measure of the quality or goodness of the hand-eye calibration THEr of the robot H, 30 in this position or pose change, and the quality or goodness of the hand-eye calibration THEr of the robot H, 30 can be quantitatively evaluated via the data processing unit 34 using this measure.

In another variant of the evaluation according to the disclosure—under the assumption that a base of the robot arm 30, in particular a base of the robot proximal to the joint 28, remains fixed in position during the position or pose changes—the hand-eye calibration THEr of the robot H, 30 can be ascertained directly from the following equation (4):

$$TAH 1 - TAH 2 = ( TBH 1 - TBH 2 ) * THEr \qquad ( 4 )$$

with TAH1 and TAH2 as the transformations of the end segment H to the local coordinate system KA of the IR tracking camera A for both poses (1st and 2nd) of the robot, TBH1 and TBH2 as the transformation of the end segment H to a local coordinate system KB of the robot base B for both poses (1st and 2nd) of the robot and THEr as the hand-eye calibration of the robot H, 30.

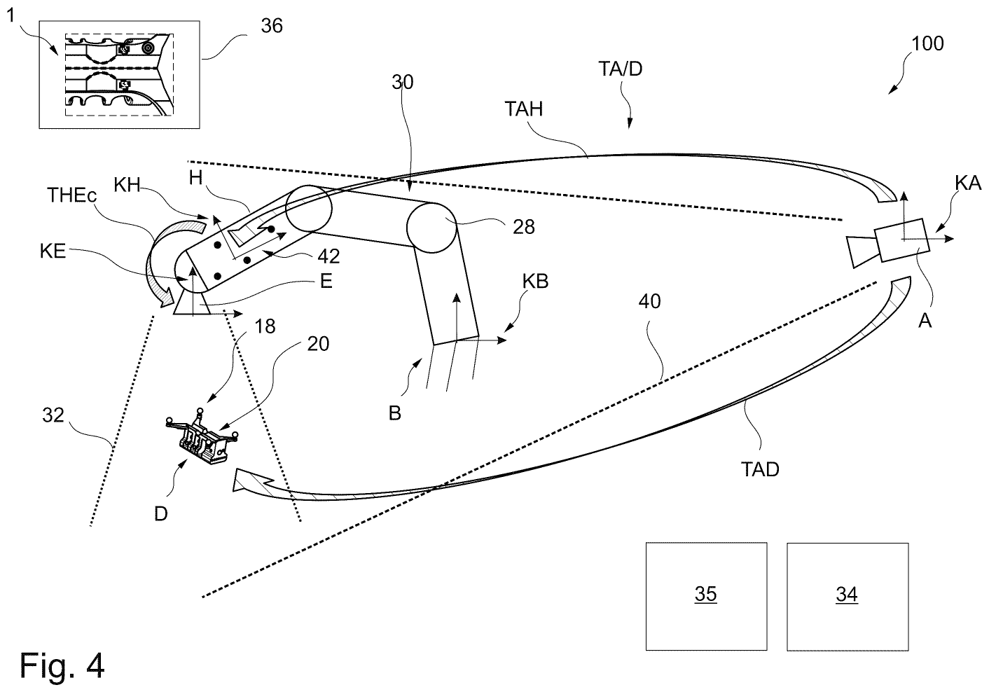

In addition to the quantitative evaluations described above, the user or operator can perform a simple, quick and clear evaluation based on an assessment of geometric features in the first image and based on geometric features of the calibration body D that are superimposed/back-projected into the first image and calculated on the basis of the calibrations, which is described in FIGS. 4 through 6. The global quality or goodness of the calibration of the assistance system 100 can be qualitatively evaluated/assessed.

As already described, according to FIG. 4, the data processing unit 34 first determines the presence of the calibration body D in the fields of view 32 and 40, or in the first and second images, whereupon it can be qualitatively evaluated. The inner and/or hand-eye calibration THEc of the optical recording unit E must be present or already carried out. The position of the calibration body D relative to the local coordinate system KE of the optical recording unit E can then be determined via the data processing unit 34.

The machine vision algorithm stored in the data processing unit 34 for execution identifies characteristic geometric features of the calibration body D and applies the combination of transformations described above to these geometric features. In this way, the data processing unit 34 calculates virtual representations of the geometric features, which are then projected back into the first image by controlling the display unit 36 by the data processing unit 34. This creates an overlay of the real geometric features 20, 22, 24 with their virtual calculated representations 20′, 22′, 24′.

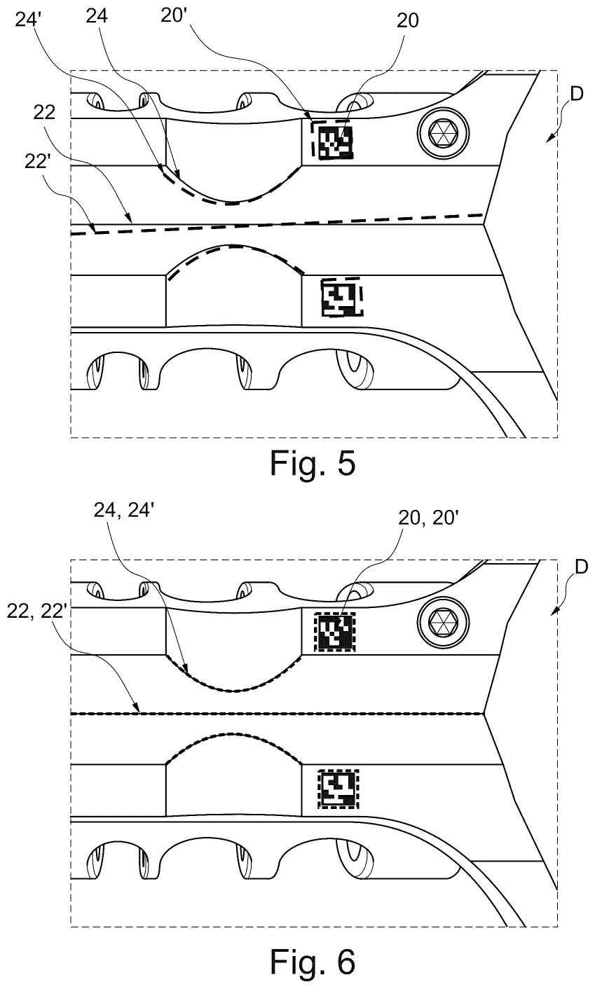

FIG. 5 shows such back projections with comparatively poor, global calibration of the assistance system 100, which can be seen very clearly recognizable for the surgeon/user based on the positional deviation of the back projected representations 20′, 22′, 24′ compared to their originals 20, 22, 24 shown in the first image.

Therefore, the surgeon/user can verify clearly whether the calibration of the assistance system meets his/her requirements.

In the case of a well-calibrated assistance system 100, the back-projected representations 20′, 22′, 24′ are in synch with their originals 20, 22, 24 shown in the first image, as shown in FIG. 6.

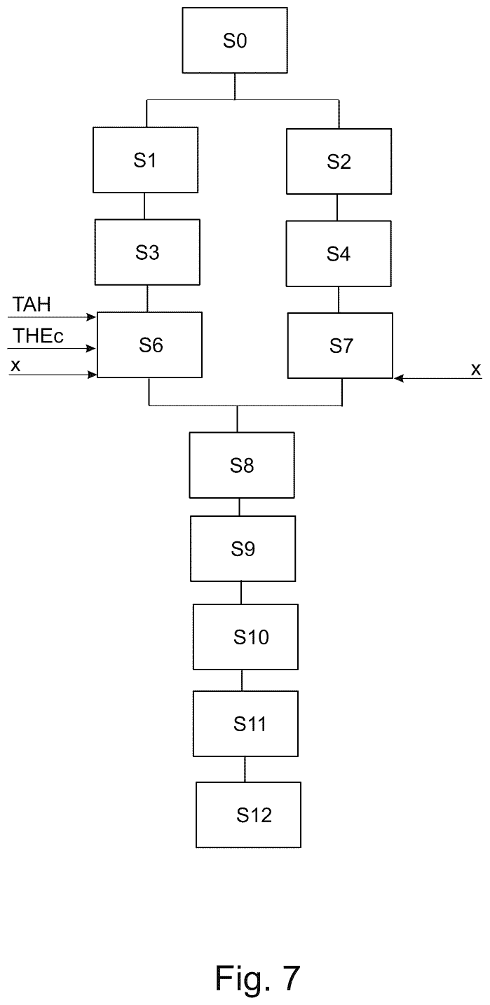

FIG. 7 shows a flow diagram of a preferred exemplary embodiment of a method according to the disclosure for evaluating at least one calibration of the assistance system 100.

The method has a first step S0 Placement of the calibration body D in the fields of view 32, 40 of the optical recording unit E and the IR tracking unit A according to FIGS. 2 through 4.

The following are steps S1 Creating and providing the first image of the optical marker 20 of the calibration body D by the optical recording unit E and S2 Creating and providing the second image of the IR marker arrangement 18 of the calibration body D and the IR rigid body 42 by the IR tracking unit A.

According to the disclosure, the steps S3 determine the first transformation TED of the calibration body D relative to the first coordinate system KE of the optical recording unit E, based on the first recording, and S4 determine the second transformation TAD of the calibration body D relative to the second coordinate system KA of the IR tracking unit A, based on the second recording, and at the end of the method, S5 evaluate the at least one calibration TEHc, TEHr of the assistance system 100 at least on the basis of the first transformation TED and the second transformation TAD, each by the data processing unit 34.

First, in a step S6, a link TA/D of several transformations is determined, namely from a rigid body transformation TAH of the rigid body 42 relative to the second coordinate system KA of the IR tracking unit A, from a transformation TEHc of the optical recording unit E relative to the rigid body 42 and from the first transformation TED of the calibration body D relative to the first coordinate system KE. The transformation TEHc of the optical recording unit E is referred to as its hand-eye calibration and is included in the method in a predetermined, in particular estimated, form. In step S6, the value of this link is also determined for a spatial point x, preferably a body point of the calibration body D, shown in the first image and in the second image.

In a step S7, the second transformation TAD is determined for the spatial point x.

In step S8, the Euclidean distance E(x) is determined based on the difference between the link TA/D determined in step S6 for the spatial point x and the second transformation TAD determined in step S6 for the spatial point x as a measure of the quality or quality of the hand-eye calibration TEHc of the optical recording unit E, and in step S9 the quantitative evaluation of the quality or quality of the hand-eye calibration TEHc of the optical recording unit E based on the Euclideic distance E(x).

Based on this, steps S10 determine the Euclidean distance E(x) for different layers or poses of the robot H, 30; S11 determines a difference of the Euclidean distances E(x) of the layers or poses as a measure of a hand-eye calibration THEr of the robot H, 30 and S12 quantitative evaluation of the hand-eye calibration of this base of the robot.

In addition, the qualitative evaluation of the global calibration of the assistance system described above can be carried out by the user or surgeon as needed.

### LIST OF REFERENCE SIGNS

1 Medical instrument
2 Longitudinal axis
4 Base body
6 Top side
8 Edge side
10 Front side
12 Through-recess
14 Through-recess
16 Boom
18 IR marker
20 Optical marker
22 Longitudinal groove
24 Transverse groove
28 Joint
30 Robot arm
32 Visual field of view optical recording unit
36 Display unit
40 Infrared tracking unit field of view
42 Rigid body
100 Medical assistance system
A IR tracking unit
B Robot base
D Calibration body
H Robot end segment
TAD, TAH, TED Transformation
TA/D Linking of transformations
THEc, THEr Hand-eye calibration
S0KA, KB, KE, KH Coordinate system
S0 Step Placement
S1 Step: Arrangement
S2 Step: Creation of the recording
S3 Step: Provision of the recording
S4 Step: Recording the first reference system
S5 Step: Determination
S6 Step: Recording of the second reference system
S7 Step: Determination
S7.1 Step: Determination based only on the first reference system
S7.2 Step: Determination based only on the second reference system
S7.3 Step: Determination based on both reference systems
S8 Step: Ascertaining of sufficient detection of first reference system
S9 Step: Ascertaining of sufficient detection of second reference system
S10 Determination of the position and/or orientation of the distal end section

1. A calibration body for a medical assistance system, the calibration body comprising:
an optical marker as a first marker, the optical marker adapted to be captured by a movably arranged optical recording unit of the medical assistance system with a first recording, based on a first position of the calibration body relative to the first optical recording system is determinable; and
an IR marker arrangement as a second marker, the IR marker arrangement comprising at least three IR markers adapted to be detected by an IR tracking unit of the medical assistance system with a second recording, a second layer of the calibration body relative to the IR tracking unit being determinable based on the second recording in order to enable an evaluation of at least one calibration of the medical assistance system by the optical marker and the IR marker arrangement.

2. The calibration body according to claim 1, wherein the optical marker is formed as an optical pattern at least in sections from a flat bar code, grid code, QR code, laser print or imprint or from a flat anodizing or from an engraving.

3. The calibration body according to claim 1, wherein the IR marker arrangement has four infrared reflector bodies and/or infrared LEDs.

4. The calibration body according to claim 1, wherein four optical patterns are arranged in a first plane and four IR markers are arranged as an IR marker arrangement in a second plane, wherein the first plane is parallel to the second plane and the optical patterns are arranged inside the IR marker in a plan view of the calibration body.

5. The calibration body according to claim 1, wherein the calibration body has a base body and the optical marker extends to a surface of the base body, and the IR markers are each arranged on an extension or boom or arm projecting from the base body.

6. The calibration body according to claim 5, wherein the optical marker is supplemented by optically detectable geometric features of the calibration body.

7. The calibration body according to claim 5, wherein the base body is thickened in an anvil shape on its rear side with respect to the markers in order to form an undercut, and on its side having the markers has crossed grooves or channels as geometric features and the IR markers are arranged protruding from a surface of this side.

8. A medical assistance system for a surgical intervention, the medical assistance system comprising:
a calibration body according to claim 1;
an optical recording unit, which is connected to a movable end segment of a robot arm of a robot and is adapted to create and provide an optical first recording;
an IR tracking unit, which is adapted to create and provide a second image and to track an IR rigid body fixed to the end segment; and
a data processing unit,
the data processing unit adapted to determine, based on the first recording of the optical marker of the calibration body, a first position of the calibration body relative to the first, optical recording unit, to determine, based on the second recording of the IR marker arrangement of the calibration body, a second layer of the calibration body relative to the IR tracking unit, to determine, and, based on the second image of the IR rigid body, a rigid body transformation of the IR rigid body relative to a second coordinate system of the IR tracking unit, and, with an increase in a hand-eye transformation of the optical recording unit relative to the rigid body, to determine a link of the rigid body transformation with the hand-eye transformation of the first optical recording unit and with the first transformation, and to compare this link with the second transformation in order to thereby carry out an evaluation of at least one calibration.

9. The medical assistance system according to claim 8, wherein the data processing unit is adapted to determine the link and to determine the second transformation for a spatial point imaged in the first image and in the second image, and that the data processing unit is further adapted to determine a Euclidean distance) based on a difference between the determined link and the determined second transformation as a measure of the hand-eye transformation of the optical recording unit relative to the rigid body, and to indicate a quantitative evaluation of the hand-eye calibration of the optical recording unit by means of this measure.

10. The medical assistance system according to claim 9, wherein the data processing unit is adapted to determine the Euclidean distance) for at least two different positions or poses of the robot, and that the data processing unit is adapted to determine a difference in the Euclidean distances) of the positions or poses as a measure of the hand-eye transformation of the robot, and to indicate a quantitative evaluation of the hand-eye calibration of the robot by means of this measure.

11. The medical assistance system according to claim 10, wherein a display unit is provided, and that the data processing unit is adapted to detect a presence of the calibration body in a respective field of view of the optical recording unit and the IR tracking unit or in the first recording and in the second recording,
in the presence of determining the first transformation of the calibration body relative to the first coordinate system of the optical recording unit, and
based on at least the first transformation to back-project geometric features of the calibration body into a plane of the first recording, and
to control the display unit to display the first recording together with the back-projected geometric features,
so that a user can qualitatively evaluate a calibration of the medical assistance system depending on a deviation of the back-projected geometric features from the geometric features that can be recognized in the first recording.

12. A computer-implemented method for evaluating a calibration of a medical assistance system according to claim 8, the method comprising the steps of:
producing and providing a first recording of an optical marker of the calibration body by the optical recording unit;
creating and providing a second recording of an IR marker arrangement of the calibration body and an IR rigid body rigidly attached to the end segment by the IR tracking unit;
determining a first position of the calibration body relative to the first optical recording unit, based on the first recording, by a data processing unit in which a machine vision algorithm is stored for execution;
determining a second position of the calibration body relative to the IR tracking unit, based on the second recording, by the data processing unit; and
evaluating at least one calibration of the assistance system based on at least the first transformation and the second transformation.

13. The computer-implemented method according to claim 12, further comprising the steps of:
determining a link from a rigid body transformation of the rigid body relative to the second coordinate system, a hand-eye transformation of the optical recording unit relative to the rigid body, and the first transformation of the calibration body relative to the first coordinate system, by the data processing unit for a spatial point recorded in the first image and in the second image;
determining the second transformation for the spatial point, by the data processing unit; and
determining a Euclidean distance based on a difference between the determined link and the determined second transformation as a measure of a quality or goodness of a hand-eye calibration of the optical recording unit; and
evaluating quantitatively the quality or goodness of the hand-eye calibration of the optical recording unit based on the Euclidean distance.

14. The computer-implemented method according to claim 13, further comprising the steps of:
determining the Euclidean distance for layers or poses of the robot, by the data processing unit;
determining a difference of Euclidean distances of the layers or poses as a measure of a hand-eye calibration of the robot, by the data processing unit; and
evaluating quantitatively the hand-eye calibration of the robot based on the difference of the Euclidean distances of the layers or poses.

15. A computer-readable storage medium comprising instructions which, when executed by a computer, cause the computer to perform the computer-implemented method according to claim 12.
